(19) United States
(12) Patent Application Publication
(10) Pub. No.: US 2014/0309589 A1
(43) Pub. Date: Oct. 16, 2014

(54) FLUID INJECTION DEVICE AND CLOGGING DETECTING METHOD

(71) Applicant: SEIKO EPSON CORPORATION, Tokyo (JP)

(72) Inventors: Yoshihiko MOMOSE, Shiojiri-shi (JP); Tsutomu MIYAMOTO, Shiojiri-shi (JP)

(73) Assignee: SEIKO EPSON CORPORATION, Tokyo (JP)

(21) Appl. No.: 14/247,634

(22) Filed: Apr. 8, 2014

(30) Foreign Application Priority Data
Apr. 10, 2013 (JP) 2013-081890

(51) Int. Cl. G01M 99/00; A61M 5/142

(52) U.S. Cl. CPC G01M 99/00; A61M 5/142; A61M 2205/70
USPC 604/151; 73/37

(57) ABSTRACT

A fluid injection device includes a pump which causes fluid to fluctuate, a fluid receiving member which includes a space for receiving at least a part of the fluctuated fluid, and a penetration hole which penetrates to the space, a film-like member which is provided on the penetration hole, and a sensor which detects a change of the film-like member. Rigidity of the fluid receiving member is higher than rigidity of the film-like member.

### BACKGROUND

1. Technical Field

The present invention relates to a fluid injection device which injects fluid, and a clogging detecting method of the fluid injection device.

2. Related Art

An insulin pump for injecting insulin into a living body has been used in practice. A fluid injection device such as the insulin pump is fixed to a living body such as a human body, to periodically inject fluid to a living body such as a human body based on a preset program.

JP-A-2010-48121 discloses a micropump including a transport mechanism configured with a cam, a finger, and a tube, and a reservoir (FIG. 5).

If clogging occurs in the fluid injection device such as the micropump, it is difficult to inject the fluid to an injection target. Accordingly, it is preferable to detect the clogging of the fluid with high sensitivity.

### SUMMARY

An advantage of some aspects of the invention is to detect clogging of fluid in a fluid injection device with higher sensitivity.

An aspect of the invention is directed to a fluid injection device including: a pump which causes fluid to fluctuate; a fluid receiving member which includes a space for receiving at least a part of the fluctuated fluid, and a penetration hole which penetrates to the space; a film-like member which is provided on the penetration hole; and a sensor which detects a change of the film-like member, in which rigidity of the fluid receiving member is higher than rigidity of the film-like member.

The other configurations will be made clear with reference to the present specification and accompanied drawings.

### BRIEF DESCRIPTION OF THE DRAWINGS

The invention will be described with reference to the accompanying drawings, wherein like numbers reference like elements.

FIG. 1 is an overall perspective view of a micropump.

FIG. 2 is a diagram of the separated micropump.

FIG. 3 is a projection top view of the micropump.

FIG. 4 is a cross-sectional view of the micropump.

FIG. 5 is a perspective view of an inner portion of a main body.

FIG. 6 is a perspective view of a rear surface of the main body.

FIG. 7 is an exploded perspective view of a cartridge.

FIG. 8 is a perspective view of a rear surface of a cartridge base.

FIG. 9 is a perspective view of a rear surface of the micropump.

FIG. 10 is an explanatory diagram of a rotary finger pump.

FIG. 11 is a cross-sectional view taken along line B-B of FIG. 3.

FIG. 12 is a cross-sectional view taken along line C-C of FIG. 3.

FIG. 13 is a cross-sectional view taken along line B-B of FIG. 3.

FIG. 14 is a cross-sectional view taken along line C-C of FIG. 3.

FIG. 15 is a graph showing sensitivity characteristics in a case of using a pressure detecting member of the embodiment.

FIG. 16 is a graph of sensitivity characteristics of a reference example.

FIG. 17A is a first perspective view of a rotary finger pump of the other embodiment and FIG. 17B is a second perspective view of a rotary finger pump of the other embodiment.

### DESCRIPTION OF EXEMPLARY EMBODIMENTS

At least the following aspects are made clear with reference to the specification and the accompanied drawings. There is provided a fluid injection device including: a pump which causes fluid to fluctuate; a fluid receiving member which includes a space for receiving at least apart of the fluctuated fluid, and a penetration hole which penetrates to the space; a film-like member which is provided on the penetration hole; and a sensor which detects a change of the film-like member, in which rigidity of the fluid receiving member is higher than rigidity of the film-like member.

As described above, since the rigidity of the fluid of the fluid receiving member is higher than the rigidity of the film-like member, in a case where clogging occurs downstream of the fluid receiving member, the fluid moves to the film-like member provided on the penetration hole of the fluid receiving member in a concentrated manner. It is possible to detect clogging with higher sensitivity by detecting a change of the film-like member.

In the fluid injection device, it is preferable that the space in the fluid receiving member be a communication hole through which the fluid flows in a flow direction.

By doing so, since it is possible to set the space in the fluid receiving member as a part of a flow path of the fluid, it is possible to have a miniaturized fluid injection device.

It is preferable that rigidity of a tube connected to the fluid receiving member be higher than rigidity of the film-like member.

By doing so, since the rigidity of the tube connected to the fluid receiving member is higher than the rigidity of the film-like member, in a case where clogging occurs on the tube connected to a downstream side of the fluid receiving member, the fluid moves to the film-like member in a concentrated manner. It is possible to detect clogging with higher sensitivity by detecting the change of the film-like member.

It is preferable that the sensor be a pressure sensor.

By doing so, it is possible to detect the change of the film-like member.

It is preferable that a plate-like member which comes in contact with the pressure sensor be provided on the film-like member.

By doing so, it is possible to press the pressure sensor with the plate-like member even in a case where the film-like member is deformed with pressure of the fluid. It is possible to detect clogging with high sensitivity.

It is preferable that the pressure sensor include a spherical member which comes in contact with the plate-like member to transfer a force to a semiconductor force sensor in the pressure sensor.

By doing so, since the spherical member theoretically comes in contact with one part of the plate-like member, it is possible to detect the change of the film-like member with excellent sensitivity.

It is preferable that the film-like member contain an elastomer.

By doing so, it is possible to detect clogging with high sensitivity using the film-like member having flexibility.

At least the following configurations will be made clear with the specification and the accompanied drawings. That is, as one aspect, the invention provides a clogging detecting method of a fluid injection device including a fluid receiving member which includes a space for receiving at least a part of fluctuated fluid and a penetration hole which penetrates to the space, and a film-like member which is provided on the penetration hole, in which rigidity of the fluid receiving member is higher than rigidity of the film-like member, the method including: operating a pump to cause the fluid to flow; detecting a change of the film-like member; and detecting clogging on a downstream side of the fluid receiving member based on the change of the film-like member.

As described above, since the rigidity of the fluid receiving member is higher than the rigidity of the film-like member, in a case where clogging occurs downstream of the fluid receiving member, the fluid moves to the film-like member provided on the penetration hole of the fluid receiving member in a concentrated manner. It is possible to detect clogging with higher sensitivity by detecting the change of the film-like member.

### Embodiment

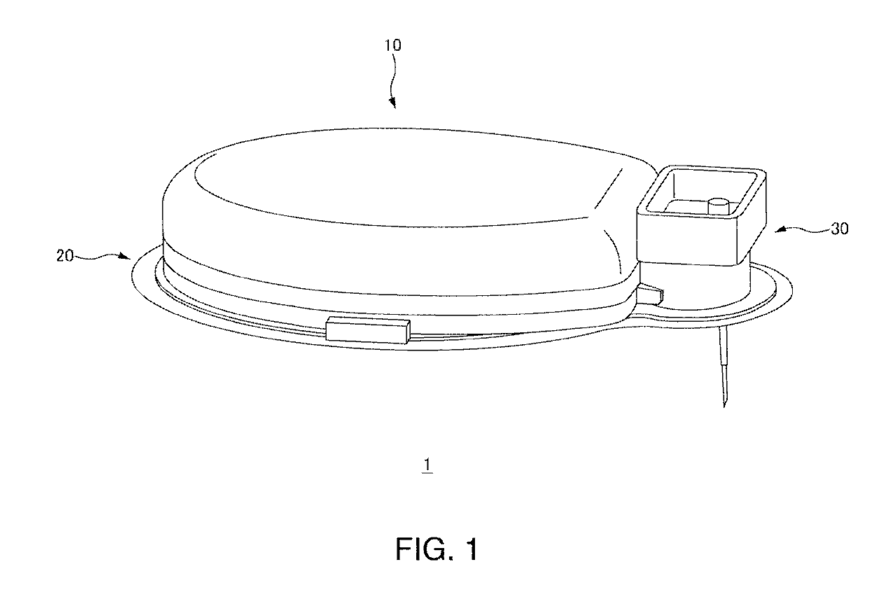

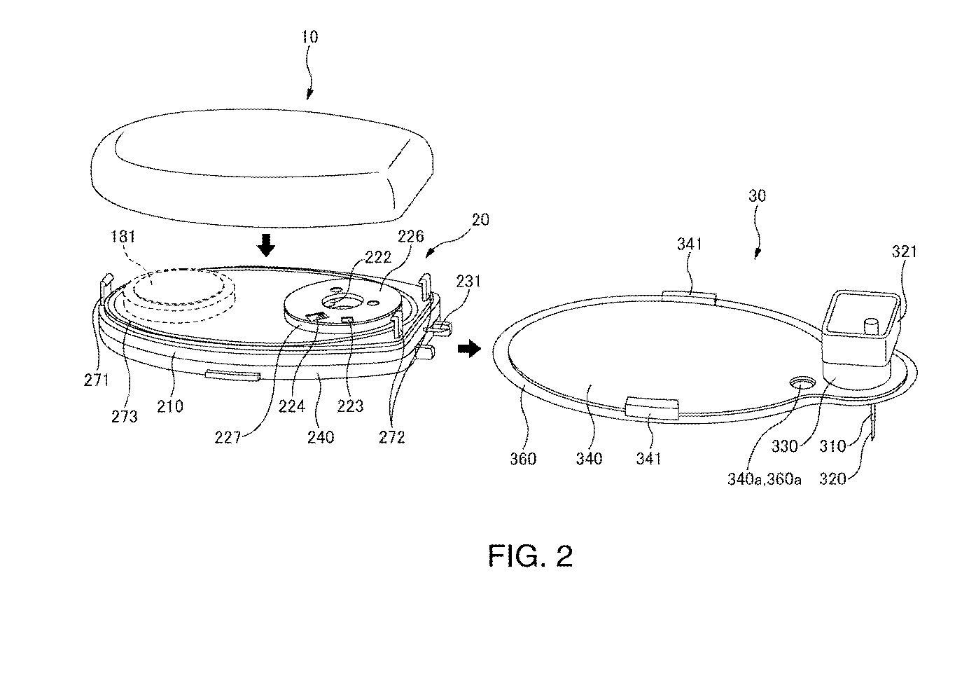

FIG. 1 is an overall perspective view of a micropump 1. FIG. 2 is a diagram of the separated micropump 1. The micropump 1 includes a main body 10, a cartridge 20, and a patch 30. These three components can be separated as shown in FIG. 2, but are integrally assembled as shown in FIG. 1 when used. The micropump 1 of the embodiment is adhered to a living body and is suitably used for regular injection of insulin.

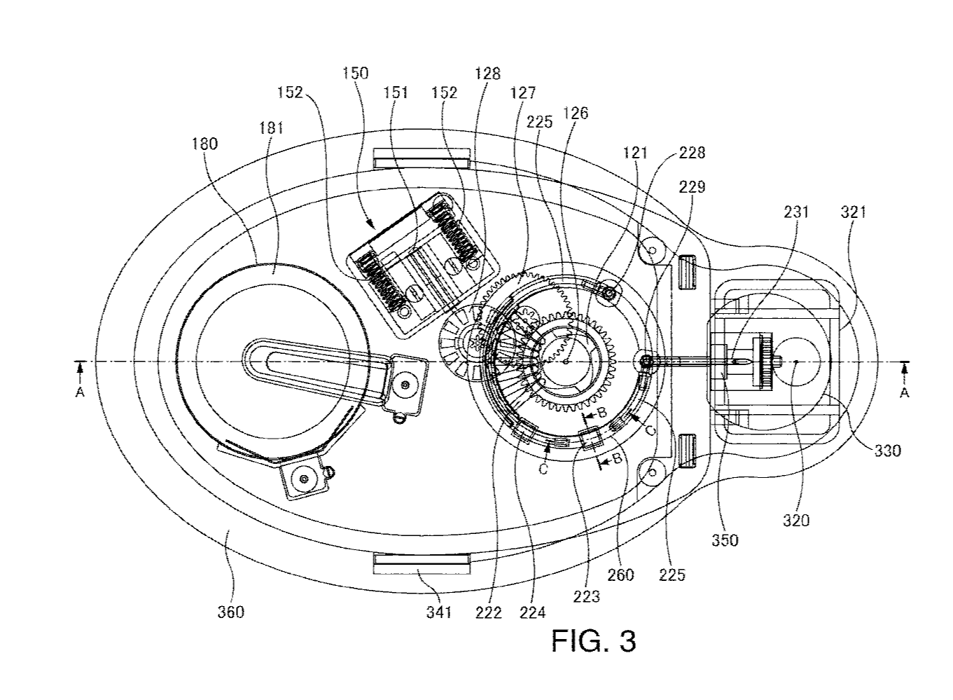

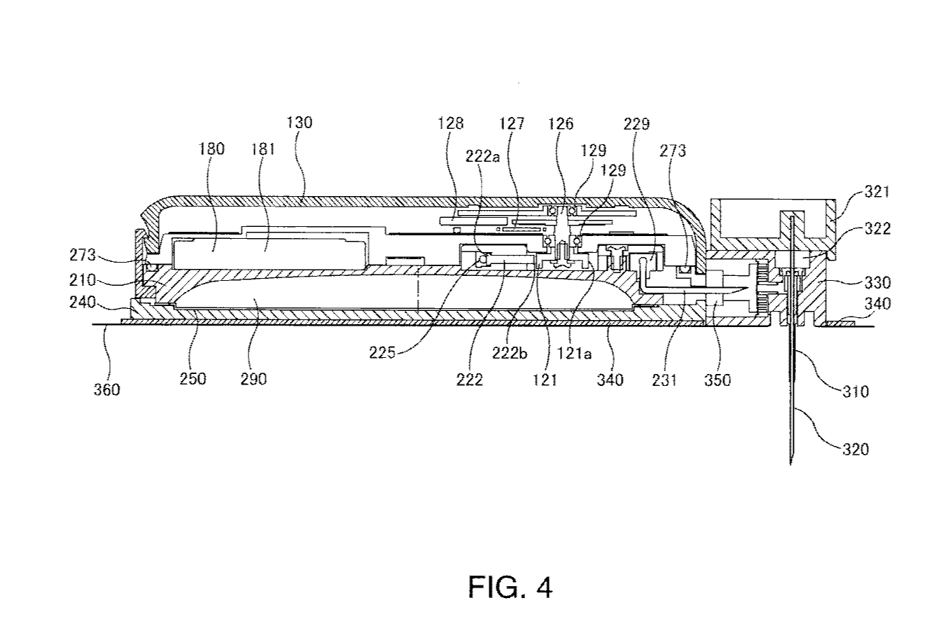

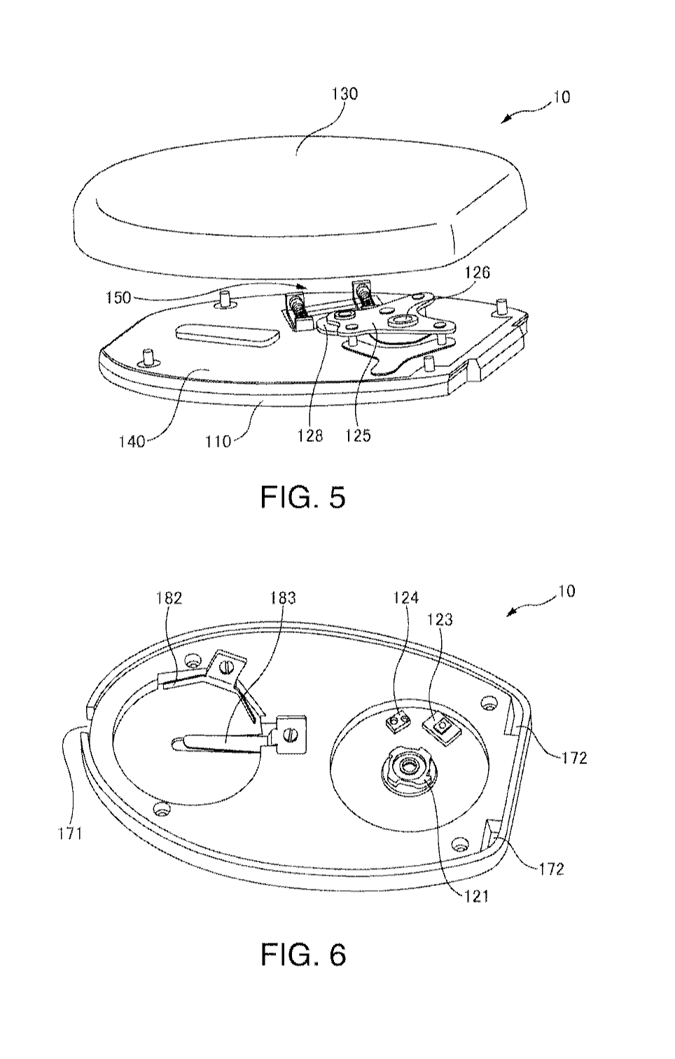

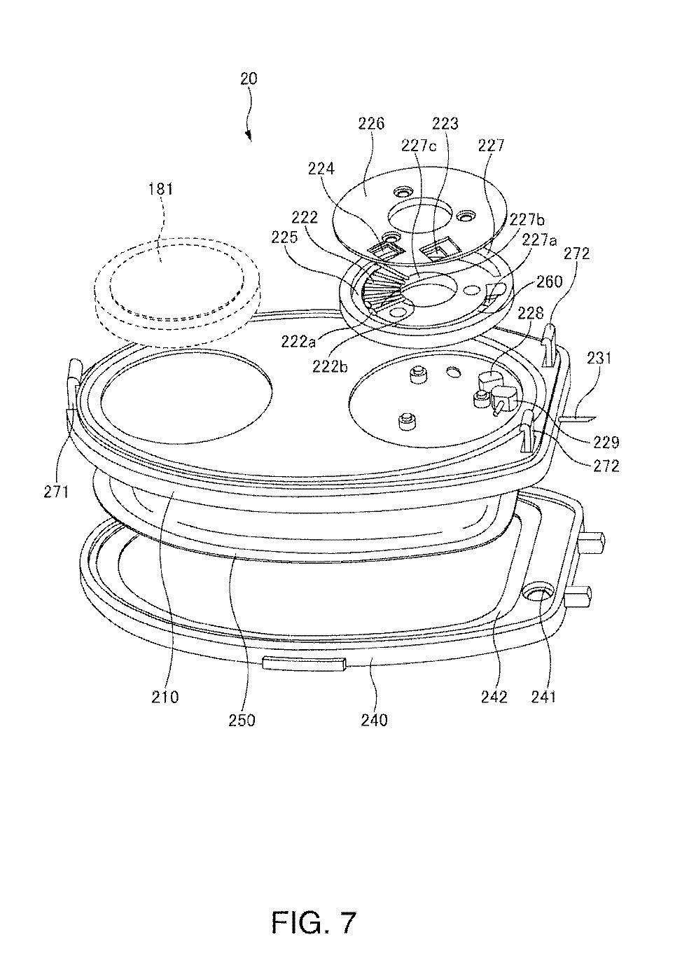

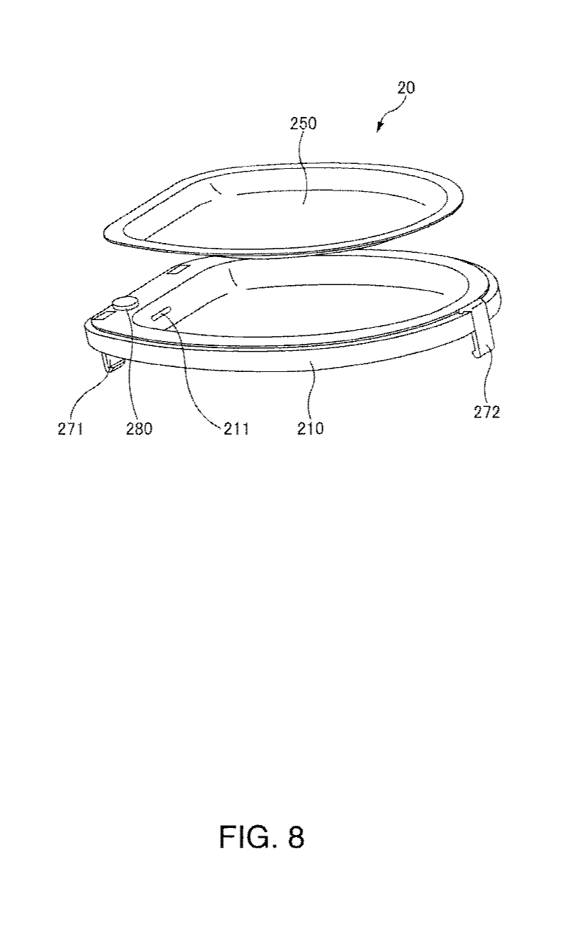

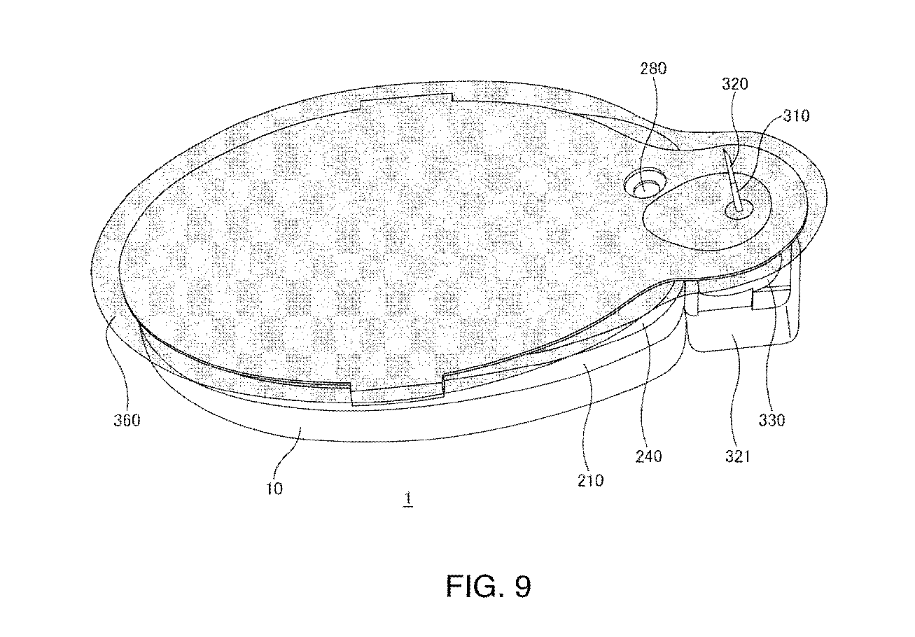

FIG. 3 is a projection top view of the micropump 1. FIG. 4 is a cross-sectional view of the micropump 1. That is, FIGS. 3 and 4 are diagrams when the main body 10, the cartridge 20, and the patch 30 are assembled. FIG. 5 is a perspective view of an inner portion of the main body 10. FIG. 6 is a perspective view of a rear surface of the main body 10. FIG. 6 is a diagram showing a rear surface of FIG. 5 described above. FIG. 7 is an exploded perspective view of the cartridge 20. FIG. 8 is a perspective view of a rear surface of a cartridge base 210. FIG. 9 is a perspective view of a rear surface of the micropump 1.

Hereinafter, each unit of the micropump 1 will be described with reference to FIGS. 1 to 9 described above. First, each unit of the main body 10 will be described.

The main body 10 includes a main body base 110, units configured on the main body base 110, and a main body case 130. Each unit on the main body base 110 is covered and protected by the main body case 130.

The main body 10 includes a circuit board 140 which is configured on the main body base 110. The circuit board 140 is an electronic substrate for performing control of a piezoelectric motor 150 and the like according to a program and the like. In addition, the main body includes the piezoelectric motor 150. The piezoelectric motor 150 is a motor for applying a rotation driving force to a cam 121 which will be described later.

The piezoelectric motor 150 includes a plate-like member 151 and a pair of springs 152 (FIG. 3). The springs 152 cause the plate-like member 151 to be biased towards a rotor wheel 128 with an elastic force thereof. The plate-like member 151 is biased towards the rotor wheel 128 as described above and a distal end portion thereof comes in contact with a circumferential surface of the rotor wheel 128.

The plate-like member 151 is a member configured to have a layer shape. The plate-like member 151 includes a piezoelectric layer and two electrodes, and changes a shape thereof by a change in voltage applied to the two electrodes. For example, longitudinal vibration and bending vibration are alternately repeated by the applied voltage. The longitudinal vibration changes a length of the plate-like member 151 in an axis direction thereof, and the bending vibration changes the plate-like member to an approximately S-shaped member. By alternately repeating the vibration, the rotor wheel 128 is rotated in a predetermined direction.

The rotor wheel 128 includes pinions which integrally rotate on different positions in a height direction of the micropump 1, and the pinions are engaged with gears of an intermediate wheel 127 to rotate the intermediate wheel 127. The intermediate wheel 127 also includes pinions which integrally rotate on different positions in the height direction of the micropump 1, and the pinions are engaged with gears which integrally rotate with an output shaft 126. Each shaft of the rotor wheel 128, the intermediate wheel 127, and the output shaft 126 is rotatably fixed by a wheel train base 125 which is fixed to the main body 10.

A cam 121 is also integrally and rotatably fixed to the output shaft 126 which is pivotally supported by a bearing 129. The cam 121 is also rotated with the rotation of the output shaft 126. Accordingly, power generated from the piezoelectric motor 150 is transferred to the cam 121.

As shown in FIG. 6, a hook 171 is provided on a front portion of the main body 10, and two hook insertion ports 172 are provided on a rear portion thereof. A fixed hook 271 of the cartridge 20 is engaged with the hook 171, and fixed hooks 272 are engaged with the hook insertion ports 172, and therefore it is possible to fix the cartridge 20 to the main body 10 (FIGS. 2 and 4).

At that time, since a packing 273 is fit to a groove portion on an outer periphery of an upper surface of the cartridge base 210, when the main body 10 and the cartridge 20 are fixed to each other, it is possible to seal the main body and the cartridge so that liquid or the like does not permeate the space formed by the main body and the cartridge.

The main body 10 includes a clogging detecting element 123 and an air bubble detecting element 124 on the rear surface thereof (FIG. 6). The clogging detecting element 123, for example, includes a pressure sensor. In addition, the air bubble detecting element 124, for example, includes an optical sensor. The optical sensor emits light to a tube 225 and detects reflection light thereof. Then, a difference between reflection light when liquid occupies the inner portion of the tube 225 and reflection light when air bubbles are generated can be detected. Accordingly, it is possible to determine whether or not air bubbles are generated in the tube 225.

The main body 10 includes a secondary battery accommodation unit 180 on the rear surface thereof (FIG. 6). The secondary battery accommodation unit 180 includes a battery positive terminal 182 and a battery negative terminal 183, a secondary battery 181 is inserted into the secondary battery accommodation unit, and accordingly, predetermined power supply can be performed in each unit of the main body 10.

Next, the cartridge 20 will be described.

The cartridge 20 includes the cartridge base 210, a cartridge base retainer 240, and units configured on the cartridge base. As will be described later, the cartridge base 210 configures a storage unit 290 with a reservoir film 250.

The cartridge base 210 of the cartridge 20 includes a finger unit 220 on an upper surface thereof. The finger unit 220 includes a finger base 227, fingers 222, the tube 225, and a finger retainer 226. A suction connector 228 and a discharge connector 229 are provided on the upper surface of the cartridge base 210. The suction connector 228 is a connector for sucking liquid to the finger unit 220 and the discharge connector 229 is a connector for discharging liquid from the finger unit 220.

A plurality of grooves are formed on the finger base 227, and the suction connector 228 and the discharge connector 229 are inserted into the grooves. A tube guide groove 227a which guides the tube 225 is formed on the finger base 227 in an arc-like shape, and accommodates the tube 225 and the like. The tube 225 is tightly connected to the suction connector 228 and the discharge connector 229.

A plurality of finger guides 227b are formed in the arc of the tube guide groove 227a. Each of the finger guides 227b accommodates the finger 222. Accordingly, a distal end portion 222a of each finger 222 is disposed to be in approximately vertical direction with respect to the tube 225.

The finger retainer 226 is fixed on the upper surface of the finger base 227 by a fixed screw (not shown). Accordingly, the finger 222 can be slidably moved only in a direction along the finger guide 227b.

As described above, since the finger 222 and the tube 225 are provided on the cartridge 20 side, although diameters of the tube 225 are set to be different from each other, it is possible to provide the cartridge 20 with fingers 222 having lengths in accordance with the diameters of the tube. Accordingly, although a size of the cam 121 is set to a standardized size, it is possible to appropriately dispose a cam surface 121a of the cam 121 in a position in which the cam surface comes in contact with a rear end portion 222b of the finger 222.

A clogging detecting window 223 and an air bubble detecting window 224 are provided on the finger retainer 226. When the main body 10 and the cartridge 20 are assembled, the clogging detecting element 123 detects clogging of liquid through the clogging detecting window 223. The air bubble detecting element 124 detects presence or absence of air bubbles in the tube 225 through the air bubble detecting window 224.

A patch connection needle 231 is provided on a side surface of the cartridge base 210 and causes liquid to be transmitted to the patch 30 through a patch septum 350. The patch connection needle 231 communicates with the discharge connector 229. Meanwhile, the suction connector 228 communicates with the storage unit 290 which will be described later, through a penetration hole provided on the cartridge base 210. Accordingly, liquid in the storage unit 290 can pass through the suction connector 228, the tube 225, and the discharge connector 229, and can supply the patch connection needle 231.

As shown in FIG. 4, in the embodiment, a distal end position of the patch connection needle 231 in a height direction has substantially the same height as that of the storage unit 290. By setting as described above, the liquid passes through the tube 225 and the like on the upper surface of the cartridge 20, but a difference in height itself between the distal end position of the patch connection needle 231 and a position of the storage unit 290 is small. Accordingly, since it is possible to have a small difference in potential energy, it is possible to transfer the liquid stored in the storage unit 290 to the patch connection needle 231 with small energy. Such a configuration is advantageous in a case of using the power saving type piezoelectric motor 150 described above.

The cartridge 20 includes the reservoir film 250. A vicinity of the reservoir film 250 is interposed between the cartridge base 210, and a film retaining unit 242 which is provided on the cartridge base retainer 240. Accordingly, the storage unit 290 is configured between the reservoir film 250 and the cartridge base 210 so as to store the liquid in the storage unit 290.

The reservoir film 250 may be fixed to the cartridge base 210 by welding, and the cartridge base retainer 240 and the cartridge base 210 may be fixed to each other.

The cartridge base 210 is plastic and the surface thereof on a side where the reservoir film 250 is provided has a curved surface shape. As described above, the storage unit 290 has a curved surface shape, but since the film of the reservoir film 250 is deformed depending on a remaining amount of the liquid stored in the storage unit 290, it is possible to squeeze the storage unit to extract the liquid so that the liquid does not remain in the storage unit 290. In addition, it is preferable that the reservoir film 250 at that time be subjected to curved surface machining to a shape along the curved surface shape. By doing so, although the liquid in the storage unit 290 decreases, the reservoir film 250 is deformed in accordance with the curved surface, and accordingly it is possible to squeeze the storage unit to extract the liquid so that the liquid does not remain.

The reservoir film 250 is configured with a multilayer film. At that time, it is preferable that an inner layer be polypropylene, and it is preferable that a material excellent in a gas barrier property be selected for an outer layer. The reservoir film 250 is not limited thereto, and may be a film of a thermoplastic elastomer or a film obtained by bonding a thermoplastic elastomer and a material other than the thermoplastic elastomer to each other.

In addition, a cartridge septum 280 is provided on a lower surface side of the cartridge 20 (FIG. 9). When the cartridge base 210 and the cartridge base retainer 240 are assembled, the cartridge septum 280 is inserted into a cartridge septum insertion hole 241 provided on the cartridge base retainer 240. One surface of the cartridge septum 280 is exposed to opening portions 340a and 360a of a patch base 340 and an adhesive tape 360 (FIGS. 2 and 9), and the other surface thereof communicates with a fluid inlet port 211. The fluid inlet port 211 is opened between the reservoir film 250 and the cartridge base 210. Accordingly, liquid which is injected by an injection needle or the like through the cartridge septum 280 is stored in the storage unit 290.

Next, the patch 30 will be described mainly with reference to FIG. 4.

The patch 30 includes a catheter 310, an introduction needle 320, an introduction needle folder 321, an introduction needle septum 322, a port base 330, the patch base 340, the patch septum 350, and the adhesive tape 360.

The patch septum 350 is a component for supplying liquid into the patch 30 by inserting the patch connection needle 231 thereto as will be described later. The patch septum 350 is provided on a side wall portion of the patch 30, and accordingly when the cartridge 20 is mounted towards the side surface of the patch 30, the patch connection needle 231 penetrates through the patch septum 350.

The septum such as the patch septum 350 is formed with a material in which a hole opened by penetration of a needle or the like tends to be filled (for example, silicone rubber, isoprene rubber, butyl rubber, or the like). Accordingly, although the needle is inserted into and removed from the septum, the liquid or the like is not leaked through the septum.

The catheter 310 is a tube for injecting the liquid. A part of the catheter 310 is held by the port base 330, and another part thereof is exposed to a lower side of the port base 330. When performing the injection of the liquid using the patch 30, the exposed portion of the catheter 310 is placed in the living body or the like, and the liquid is continuously injected. Accordingly, the catheter 310 is formed with a flexible material such as a fluorine resin, a polyurethane resin or the like which has excellent biocompatibility.

The introduction needle 320 is a hollow, thin and long needle-shaped member, and a shape thereof is smaller than an inner diameter of the catheter 310. The introduction needle 320 is inserted into the catheter 310 before use. A sharp end side of the introduction needle 320 is exposed downwards of the catheter 310 and the other end thereof is fixed to the introduction needle folder 321. Before use, the introduction needle 320 is inserted through the introduction needle septum 322 which is fixed to the inner portion of the port base 330.

With the configuration described above, the introduction needle 320 is extracted from the inside of the catheter 310 by extracting the introduction needle folder 321 from the port base 330, but the liquid flowing from the patch connection needle 231 is not leaked from the introduction needle septum 322 side, and flows into a living body through the catheter 310.

The patch 30 includes the patch base 340. The patch base 340 is fixed to the port base 330, includes a cartridge fixing member 341, and can cause the cartridge 20 to be fixed to the patch 30. When the cartridge 20 is connected to the patch 30, the cartridge 20 is slidably moved with respect to the patch 30 from a left side of FIG. 2. The patch connection needle 231 provided on the cartridge 20 penetrates the patch septum 350 to be inserted into the patch 30.

In addition, the patch base 340 includes the adhesive tape 360 on the lower surface thereof. The micropump 1 can be adhered to the living body.

When the main body 10 and the cartridge 20 having the configurations described above are integrally assembled, the clogging detecting element 123 is disposed on an upper portion of the clogging detecting window 223, and the air bubble detecting element 124 is disposed on an upper portion of the air bubble detecting window 224.

In addition, when the main body 10 and the cartridge 20 are assembled, the cam 121 of the main body 10 is inserted to a cam accommodation unit 227c of the finger base 227. Accordingly, the cam surface 121a of the cam 121 is disposed in a position facing the rear end portion 222b of the finger 222. The cam surface 121a comes in contact with the rear end portion 222b of the finger 222 by the rotation of the cam 121 and the finger 222 can be slidably moved.

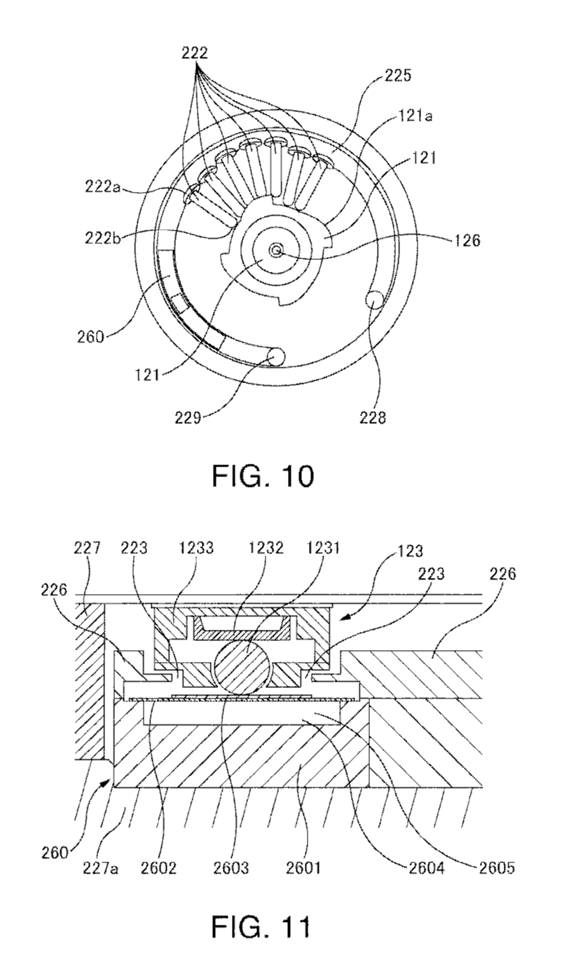

FIG. 10 is an explanatory diagram of a rotary finger pump. Four cam ridges are formed on the cam 121. Each of the cam ridges has a shape in which a height from a lowermost portion of the cam ridge to an uppermost portion thereof is gradually transitioned to be higher, and the uppermost portion is transitioned to the lowermost portion of the adjacent cam ridge. With the shape described above, if the cam 121 rotates, the distal end portions 222a of the plurality of fingers 222 sequentially presses the tube 225 in a direction from the suction connector 228 side to the discharge connector 229 side. Accordingly, it is possible to transfer the liquid in the tube 225 to the discharge connector 229 side from the suction connector 228 side.

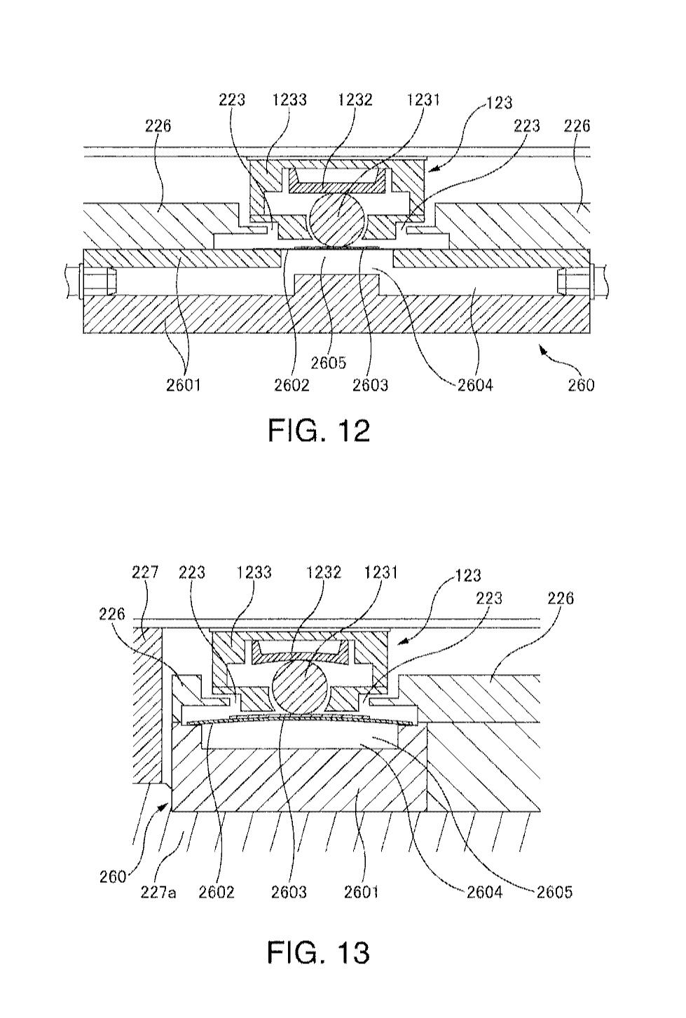

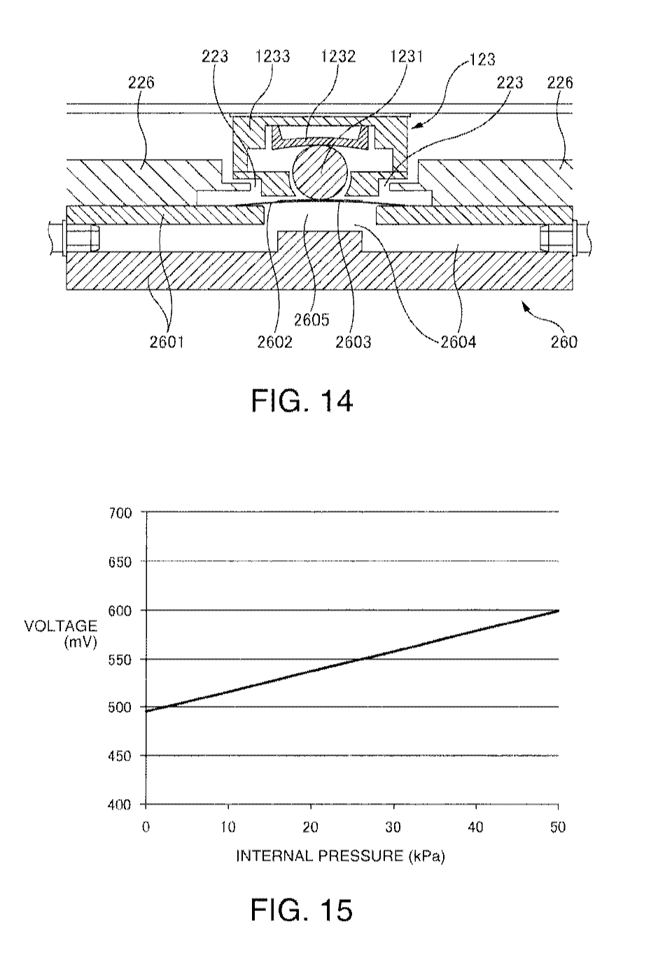

FIG. 11 is a cross-sectional view taken along line B-B of FIG. 3. FIG. 12 is a cross-sectional view taken along line C-C of FIG. 3. FIG. 13 is a cross-sectional view taken along line B-B of FIG. 3. FIG. 14 is a cross-sectional view taken along line C-C of FIG. 3. FIGS. 11 and 12 show a state before clogging occurs in the flow path of the liquid. Meanwhile, FIGS. 13 and 14 show a state when clogging occurs in the flow path of the liquid. Hereinafter, a clogging detecting unit will be described with reference to the drawings. The clogging detecting unit includes the clogging detecting element 123 (corresponding to the sensor), the clogging detecting window 223 formed on the finger retainer 226, and the pressure detecting member 260.

The clogging detecting element 123 is a pressure sensor. The clogging detecting element 123 is a pressure sensor which detects a change of a thin film 2602 which will be described later. This clogging detecting element 123 includes a semiconductor force sensor element 1232, a spherical body 1231, and an accommodation member 1233 for accommodating these. The semiconductor force sensor element 1232 is formed using a Si semiconductor substrate for detecting a force. The semiconductor force sensor element 1232 converts an applied force into an electrical signal using a piezoresistive effect, and outputs the electrical signal. The output electrical signal is transmitted to the circuit board 140. The spherical body 1231 is a component for transferring a force which is a target of measurement to the semiconductor force sensor element 1232.

The pressure detecting member 260 includes a high rigidity member 2601 (corresponding to the fluid receiving member), the thin film 2602 (corresponding to the film-like member), and a thin plate 2603 (corresponding to the plate-like member). The high rigidity member 2601 includes a communication hole 2604 which penetrates in the flow direction of the liquid and a penetration hole 2605 which penetrates to a part of the communication hole 2604 from an upper portion thereof. The high rigidity member 2601 is a member having extremely high rigidity compared to the thin film 2602, and a resin or the like is used, for example. The tube 225 also has high rigidity compared to the thin film 2602. The high rigidity member 2601 has high rigidity even compared to the tube 225.

The thin film 2602 is adhered to the high rigidity member 2601 so as to cover the penetration hole 2605. The thin film 2602 is an elastic member such as an elastomer. In addition, the thin plate 2603 is adhered to the upper surface of the center portion of the thin film 2602. The thin plate 2603 is a thin plate formed with a material such as stainless steel, and comes in contact with the spherical body 1231 to reliably transfer a change of the thin film 2602 to the clogging detecting element 123.

The pressure detecting member 260 is mounted in the tube guide groove 227a in the finger base 227. The tube 225 is fixed to an upstream end and a downstream end of the communication hole 2604 of the pressure detecting member 260. A material of the tube 225 on the downstream end side of the pressure detecting member 260 may be different from a material of the tube 225 on the upstream end side thereof.

Meanwhile, the clogging detecting element 123 is fixed to the main body 10 side as described above. If the cartridge 20 is mounted on the main body 10, the spherical body 1231 comes in contact with one point of the thin plate 2603.

In a case where clogging occurs in the flow path of the liquid when a flow operation is performed in the tube 225 by the finger unit 220, internal pressure of the high rigidity member 2601 is increased. At that time, since the thin film 2602 is an elastic body whereas the high rigidity member 2601 is a member having high rigidity, the thin film 2602 is expanded. If the thin film 2602 is expanded, the spherical body 1231 of the clogging detecting element 123 is pressed through the thin plate 2603 (FIGS. 13 and 14). At that time, the circuit board 140 monitors the applied pressure using the clogging detecting element 123. By doing so, it is possible to detect occurrence of clogging in the tube 225 when the pressure is increased more than the predetermined pressure.

If the pressure detecting member 260 is not provided, since the change in pressure due to clogging is not concentrated in the thin film 2602, it is difficult to detect the change thereof with high sensitivity. In contrast, since the rigidity of the high rigidity member 2601 of the pressure detecting member 260 is extremely high compared to the thin film 2602, in a case where clogging occurs downstream, the liquid moves to the thin film 2602 provided on the penetration hole 2605 in a concentrated manner. Accordingly, it is possible to detect clogging with higher sensitivity by detecting the change of the thin film 2602.

In the embodiment, the clogging detecting element 123 including the spherical body 1231 is used as the pressure sensor. Since the spherical body 1231 theoretically comes in contact with one point of the thin plate 2603, the clogging detecting element 123 can detect the movement of the thin film 2602 with excellent sensitivity.

The clogging detecting unit is provided downstream side of the finger 222 in the flow direction of the tube 225. This causes the fingers 222 to generate pressure in the tube 225 on the lower side thereof. That is, in the clogging detecting unit, the thin film 2602 of the pressure detecting member 260 downstream of the fingers is monitored, and the clogging detecting element 123 is set to detect the expansion of the thin film 2602 occurring when clogging occurs downstream side.

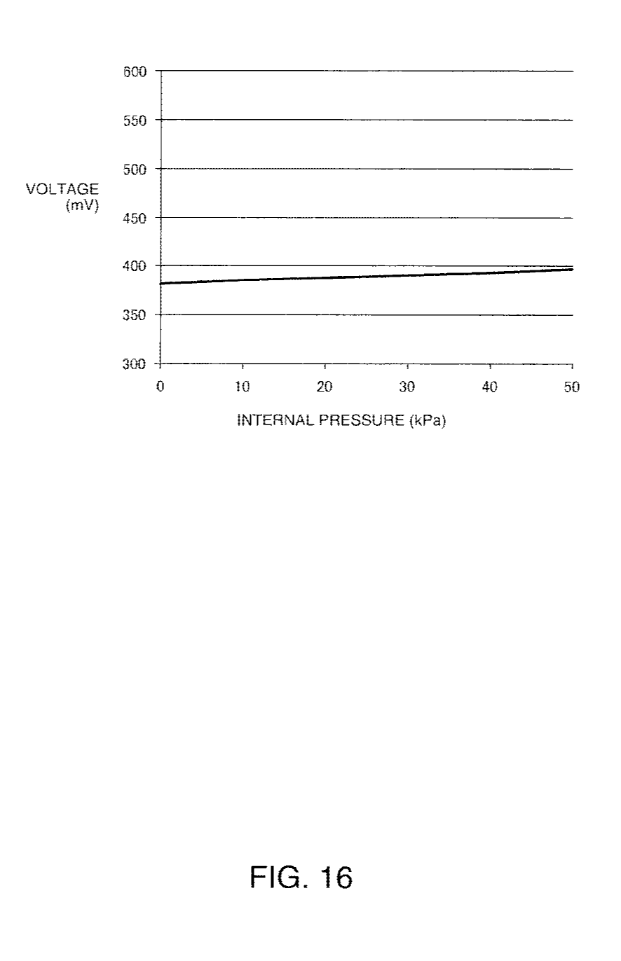

FIG. 15 is a graph showing sensitivity characteristics in a case of using the pressure detecting member 260 of the embodiment. FIG. 16 is a graph of sensitivity characteristics of a reference example. In the reference example of FIG. 16, measurement is performed so that the pressure sensor directly comes in contact with the tube through which the liquid flows.

In FIGS. 15 and 16, horizontal axis indicates internal pressure and vertical axis indicates voltage output by the pressure sensor. Herein, as the voltage increases, the pressure is detected to be high. In the graphs, as inclination is great, sensitivity with respect to pressure is high.

According thereto, inclination of FIG. 16 which is the reference example was 0.289 (mV/kPa). Meanwhile, inclination in a case of using the pressure detecting member 260 of the embodiment (FIG. 15) was 2.09 (mV/kPa). As described above, it is possible to acquire the change in pressure with higher sensitivity by using the pressure detecting member 260 of the embodiment. It is possible to detect clogging of liquid with higher sensitivity.

### Other Embodiment

In the embodiment described above, the liquid flows through the inner portion of the communication hole 2604. That is, the communication hole 2604 is a flow path of the liquid. However, according to technical ideas of the embodiment, since the device measures the internal pressure thereof, a space may be provided by the high rigidity member on a branch destination of the liquid flow path and a penetration hole which penetrates to the space may be provided. The thin film and the thin plate described above may be provided on the penetration hole, and the sensor can detect the change of the thin film. According to such configurations, it is possible to detect clogging of the liquid with higher sensitivity.

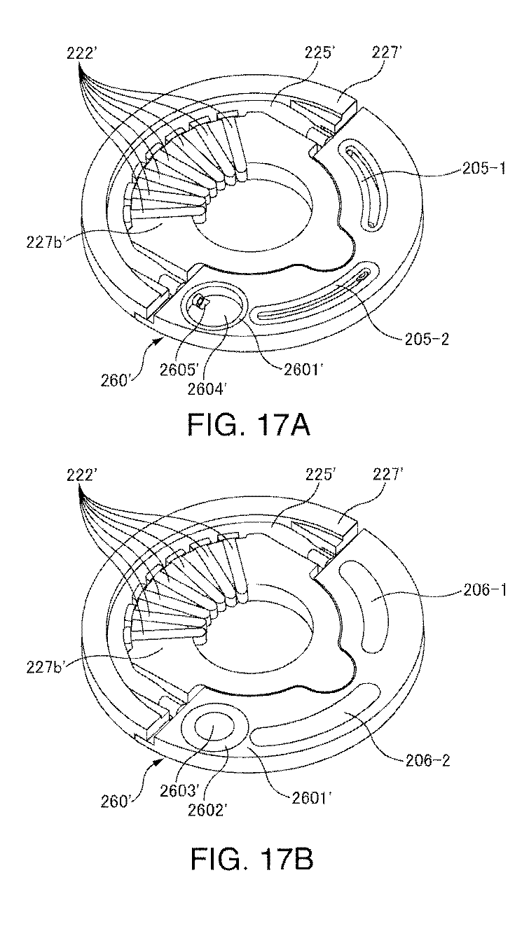

FIG. 17A is a first perspective view of a rotary finger pump of the other embodiment and FIG. 17B is a second perspective view of a rotary finger pump of the other embodiment. In the drawings, members corresponding to the above description are shown by attaching dash “′” to the reference numerals of the members described above.

In the embodiment described above, a downstream side of a pressure detecting member 260′ is connected to the tube, but herein, the downstream side of the pressure detecting member is connected to a flow path 205-2 which is formed with the same material as a high rigidity member 2601′. The flow path 205-2 is formed with the same material as the high rigidity member 2601′, and accordingly the flow path 205-2 has higher rigidity than that of a tube 225′.

In addition, an upstream of the tube 225′ is also connected to the flow path 205-1 which is formed with the same material as the high rigidity member 2601′. Since the flow path 205-1 is also formed with the same material as the high rigidity member 2601′, flow path 205-1 has higher rigidity than that of a tube 225′.

Cover members 206-1 and 206-2 which are formed with the same material as the high rigidity member 2601′ are respectively attached to the upper portions of the flow paths 205-1 and 205-2 by bonding or the like. In addition, multilayer films which are formed with different materials and have a small deformation amount may be set to the cover members 206-1 and 206-2. With such configurations, the pressure detecting member 260′ can acquire the change in pressure with higher sensitivity, and accordingly, it is possible to detect clogging of the liquid with higher sensitivity.

As shown in FIG. 17A, the pressure detecting member 260′ may include a circular communication hole 2604′. A thin film 2602′ and a thin plate 2603′ provided on an upper portion thereof may also have a circular shape (FIG. 17B).

Since it is possible to realize the miniaturized and thin micropump 1 described above and a slight amount of fluid can stably and continuously flow, the micropump is suitable to be mounted in a living body or on a surface of a living body to be used for medical treatment such as development of new drugs or drug delivery. In addition, the device may be mounted on an inner portion of various mechanical apparatuses or an outer portion thereof and may be used for transportation of fluid such as water or a saline solution, a medicinal solution, oil, perfume, ink, gas, or the like. Further, the micropump as a single body may be used for flow and supply of fluid.

The embodiments are for realizing easy understanding of the invention and are not intended to limit the invention. In the invention, modifications and improvements can be performed without departing from a gist thereof, and an equivalent thereof may be included in the invention.

The entire disclosure of Japanese Patent Application No. 2013-081890, filed Apr. 10, 2013 is expressly incorporated by reference herein.

What is claimed is:

1. A fluid injection device comprising:
a pump which causes fluid to fluctuate;
a fluid receiving member which includes a space for receiving at least a part of the fluctuated fluid, and a penetration hole which penetrates to the space;
a film-like member which is provided on the penetration hole; and
a sensor which detects a change of the film-like member,
wherein rigidity of the fluid receiving member is higher than rigidity of the film-like member.

2. The fluid injection device according to claim 1,
wherein the space in the fluid receiving member is a communication hole through which the fluid flows in a flow direction.

3. The fluid injection device according to claim 2,
wherein a cross-sectioned shape of the communication hole in a direction orthogonal to the flow direction of the fluid and a cross-sectioned shape of a flow path connected to a downstream side of the fluid receiving member in a direction orthogonal to the flow direction are different from each other.

4. The fluid injection device according to claim 1, further comprising:
a tube which is connected to a downstream side of the fluid receiving member and in which the fluid flows.

5. The fluid injection device according to claim 4,
wherein rigidity of the tube connected to the downstream side of the fluid receiving member is higher than rigidity of the film-like member.

6. The fluid injection device according to claim 1, further comprising:
a tube which is connected to an upstream side of the fluid receiving member and in which the fluid flows.

7. The fluid injection device according to claim 6,
wherein rigidity of the tube connected to the upstream side of the fluid receiving member is higher than rigidity of the film-like member.

8. The fluid injection device according to claim 1,
wherein a flow path connected to a downstream side of the fluid receiving member is formed with the same material as the fluid receiving member.

9. The fluid injection device according to claim 1,
wherein the sensor is a pressure sensor.

10. The fluid injection device according to claim 9,
wherein a plate-like member which comes in contact with the pressure sensor is provided on the film-like member.

11. The fluid injection device according to claim 10,
wherein the pressure sensor includes a spherical member which comes in contact with the plate-like member to transfer a force to a semiconductor force sensor in the pressure sensor.

12. The fluid injection device according to claim 1,
wherein the film-like member contains an elastomer.

13. A clogging detecting method of a fluid injection device including a fluid receiving member which includes a space for receiving at least a part of fluctuated fluid and a penetration hole which penetrates to the space, and a film-like member which is provided on the penetration hole, in which rigidity of the fluid receiving member is higher than rigidity of the film-like member, the method comprising:
operating a pump to cause the fluid to flow;
detecting a change of the film-like member; and
detecting clogging on a downstream side of the fluid receiving member based on the change of the film-like member.
